(19) United States
(12) Patent Application Publication
(10) Pub. No.: US 2020/0338080 A1
(43) Pub. Date: Oct. 29, 2020

(54) TOPICAL FORMULATION CONTAINING PAPAVERINE AND SILDENAFIL AND USES THEREOF

(71) Applicant: Vinod Tawar, Langley (CA)

(72) Inventor: Vinod Tawar, Langley (CA)

(21) Appl. No.: 16/859,628

(22) Filed: Apr. 27, 2020

Related U.S. Application Data
(60) Provisional application No. 62/839,717, filed on Apr. 28, 2019.

(51) Int. Cl. A61K 31/519; A61K 31/472; A61K 47/10; A61K 47/24; A61K 9/00

(52) U.S. Cl. CPC A61K 31/519; A61K 31/472; A61K 9/0014; A61K 47/24; A61K 47/10

(57) ABSTRACT

A non-invasive, non-systemic treatment for disease states resulting from perivascular diabetes and prostate disorders is disclosed. The treatment involves applying a topical formulation comprising papaverine, typically as hydrochloride USP and sildenafil typically as citrate USP to skin in the area surrounding the disease state.

### CROSS-REFERENCE TO RELATED APPLICATIONS

This application claims priority to U.S. Provisional Application No. 62/839,717, filed Apr. 28, 2019, which is hereby incorporated by reference herein in its entirety.

### FIELD OF THE INVENTION

The present invention relates generally to the field of compositions and procedures for topical applications and treatments and more specifically relates to therapeutic treatment of prostate disorders, cancer prevention and complications resulting from perivascular diseases, in particular perivascular diabetes.

### BACKGROUND

Perivascular disease is a slow and progressive circulation disorder caused by narrowing, blockage, or spasms in a blood vessel and leads to numerous and diverse disease states. Perivascular disease may affect any blood vessel outside of the heart including the arteries, veins, or lymphatic vessels resulting in tissues not receiving enough blood flow for proper function. Peripheral vascular disease is also called peripheral arterial disease.

Prostatic hyperplasia is a condition caused by the enlarging of the prostate as men age. Symptoms of prostatic hyperplasia include a frequent need to urinate as well as difficulty doing so and an inability to empty the bladder. These symptoms are due to an increase in pressure being exerted on the urethra and a thickening of bladder walls. Urinary tract, bladder and kidney problems can also be associated with prostatic hyperplasia. Other symptoms associated with prostatic hyperplasia may include obstructive voiding and erectile dysfunction. A diagnosis of prostatic hyperplasia does not indicate the presence of cancer, but both conditions may occur at the same time.

There are numerous methods for the treatment of prostatic hyperplasia and cancer prevention. In one iteration, extracts of anthocyanins and polyphenols from the Nitraria fruits are used. In another iteration, electroporation is used to induce microscopic pores in lipid cells. In still other iterations, invasive surgery or the oral induction of xenobiotic, alpha blockers or other compounds are utilized to control prostatic hyperplasia. Due to the dangers involved, many people avoid surgery while others avoid oral medications due to pre-existing medical issues and the physiological conflicts which may occur from mixing prescribed pharmaceuticals.

Hypoxia is a common microenvironmental feature of solid tumors that exists because the supply of oxygen is insufficient to meet the metabolic demand of the tumor. The poorly formed tumor blood vessels make it difficult to therapeutically increase oxygen delivery to reduce hypoxia. Hypoxia protects organisms from the detrimental effects of ionizing radiation meaning that it takes a much higher dose of radiation delivered to very hypoxic cells to get the same amount of cell kill if those cells had been fully oxygenated.

Papaverine is a cyclic adenosine monophosphate phosphodiesterase inhibitor, which leads to an increase in intracellular cyclic adenosine monophosphate, resulting in smooth muscle relaxation. Papaverine and some analogs thereof are effective mitochondrial electron transport chain complex 1 antagonists and can decrease tumor hypoxia by decreasing oxygen consumption within the tumor cells.

Papaverine hydrochloride is a potent smooth muscle relaxant and it is widely used to treat or prevent cerebral vasospasm due to its vasodilator properties. Topical use of papaverine to treat vasospasm during neurosurgical procedures was first described in 1958 and more recently has been used as a topical gel for treatment of erectile dysfunction.

Phosphodiesterase type 5 inhibitors (PDE5) are agents that are used to block the action of cGMP-specific phosphodiesterase type 5 (PDE5) on cyclic GMP. Currently, the PDE5 sildenafil, tadalafil, and vardenafil are clinically indicated for the treatment of erectile dysfunction, while sildenafil and tadalafil are also indicated for the treatment of pulmonary hypertension. The citrate salt of sildenafil, is a selective inhibitor of cyclic guanosine monophosphate (cGMP)-specific phosphodiesterase type 5 (PDE5).

Various attempts have been made to solve the problems which may be found in the related art but have been thus far unsuccessful. None of the related art, either singly or in combination, is seen to describe embodiments of the invention as claimed. Thus, a need exists for a reliable, topical combined treatment for prostate disorders, cancer prevention and complications resulting from perivascular diseases, in particular perivascular diabetes.

### BRIEF SUMMARY

It is an object of the invention to provide Topical Formulation and Methods for the Treatment of Perivascular Diseases.

In accordance with an aspect of the invention there is provided a topical formulation comprising: (i) a first active agent selected from at least one of papaverine, papaverine codecarboxylate, papaverine adenylate, papaverine teprosylate, and papaverine hydrochloride; (ii) a second active agent selected from at least one of sildenafil, sildenafil HCl, sildenafil citrate, vardenafil, avanafil, tadalafil, and alprostadil; (iii) a first compound or composition; and (iv) a second compound or composition, wherein the first compound or composition and second compound or composition are different, and each is selected from the group consisting of propylene glycol, polyvinyl alcohol; and Pluronic Lecithin Organogel, Lipoderm®, Lipmax™, Hydrogel, Medihol™, Oleabase™ Cryogel™, and Ointment Base.

In accordance with another aspect of the invention there is provided a composition, comprising: papaverine hydrochloride U.S.P.; sildenafil citrate U.S.P.; propylene glycol U.S.P.; and Pluronic Lecithin Organogel.

In accordance with another aspect of the invention there is provided a method for treating disease states associated with perivascular diabetes including: diabetic foot ulcers; peripheral neuropathy; pulmonary hypertension; cardiovascular hypertension; aneurism; Raynaud's Syndrome and combinations thereof, comprising topically applying to skin, the above compositions.

In accordance with another aspect of the invention there is provided a method for treating prostate disorders including cancer, benign prostatic hyperplasia, androgenic erectile dysfunction, prostatic hyperplasia, cancer prevention, erectile dysfunction, obstructive bladder voiding, transurethral resection of the prostate, and combinations thereof, and anorgasmic females, comprising topically applying to skin, the above compositions.

The advantages and features of the present invention will become better understood with reference to the following more detailed description and claims taken in conjunction with the accompanying FIGURE and Table.

### BRIEF DESCRIPTION OF THE SEVERAL VIEWS OF THE DRAWINGS

FIG. 1 illustrates a process for the preparation of a composition of an embodiment of the invention.

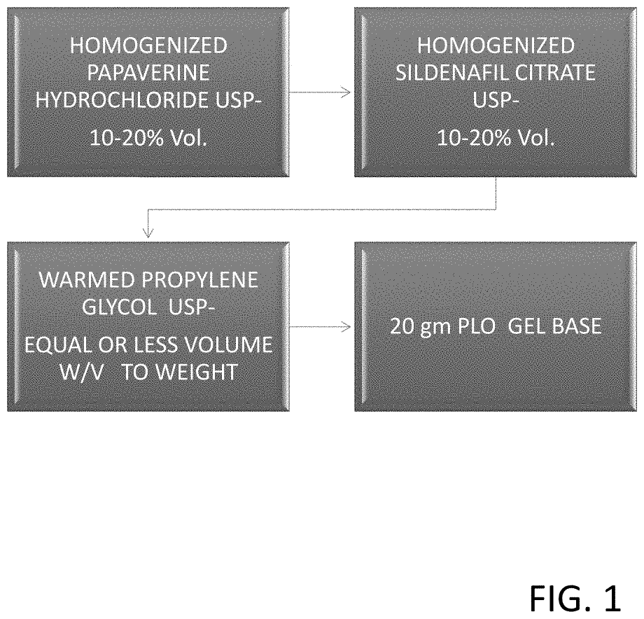

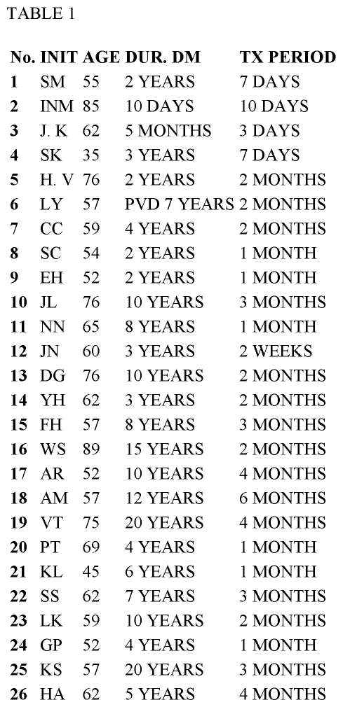

TABLE 1 illustrates demographics of patient enrollment.

### DETAILED DESCRIPTION

Compositions and methods for carrying out the invention are presented in terms of embodiments depicted within the FIGURE and Table. However, the invention is not limited to the described embodiments, and a person skilled in the art will appreciate that many other embodiments of the invention are possible without deviating from the basic concept of the invention, and that any such work around will also fall under scope of this invention. It is envisioned that other styles and configurations of the present invention can be easily incorporated into the teachings of the present invention, and the configurations shall be shown and described for purposes of clarity and disclosure and not by way of limitation of scope.

### Example 1

Composition A:

Papaverine hydrochloride U.S.P. in the range of from about 10% w/w to about 20% w/w, of the formulation.

Sildenafil citrate U.S.P. in the range of from about 10% w/w to about 20% w/w, of the formulation.

Polypropylene glycol.

PLO gel base.

The powders were homogenized to finest size and mixed well. Propylene glycol was poured over the active ingredients in powder form and warmed for the intervals of 10 seconds with 5-10 seconds pause. The mixture will dissolve to render clear solution. Finally, the PLO gel (obtained from a compounding pharmacy) was added to the solution and mixed thoroughly to a homogenous product. The product was divided into 10 gm samples and stored in a refrigerator. See Table 1.

Propylene glycol is a multifunctional component in that it acts as a solubilizer and stabilizer for the active agents. It also acts as a moisturizer. It is contemplated that numerous other pharmaceutical excipients, such as polyvinyl alcohol, could, individually or collectively function in these roles.

Numerous transdermal vehicles are known in the field and are used to deliver drugs through the skin and into the underlying tissue, joint or bloodstream. The original transdermal product was PLO gel (Pluronic Lecithin Organogel). PLO gel tends to have a tacky feel and may separate upon refrigeration.

Lipoderm® cream is an alternative transdermal base with a smooth consistency that is stable at room temperature & under refrigeration. It has been proven to deliver more drug transdermally than PLO gel′. Multiple drugs can be delivered transdermally by Lipoderm®

Lipmax™ (Lecithin & Isopropyl Palmitate) consists of mixture of lecithin and isopropyl palmitate, it may be used as a standalone vehicle or in conjunction with Pluronic 20% or 30% w/w to form a PLO Gel.

Hydrogel is a clear, non-scented silicone-in-water micro emulsion with rapid absorption properties.

Medihol™ gel base is a smooth gel intended for alcohol soluble actives. Provides cooling effect upon application while leaving minimal residue.

Oleabase™ (optionally plasticized) is a soft, odorless anhydrous ointment base exhibiting oleaginous and emollient properties for topical applications, it is an ideal vehicle for occlusive preparations and is resilient to extreme temperatures.

Ointment Base (Emulsifying) is an anhydrous base designed to protect and soften extremely dry skin. May also be used to prepare water-in-oil emulsions by adding small amounts of aqueous solution.

The primary objective of this research was to determine an effective treatment for complications of neuropathy from diabetes and preventing patients requiring amputations. The goal was achieved by trans-dermal absorption of a topical formulation having an arterial vasodilatation effect. The end result was bypassing systemic side-effects. PDE5 inhibitors have been in use systemically since 2000 for the treatment of complications resulting from diabetes e.g. erectile dysfunction, chronic kidney disease, etc. on the principle of their vasodilatation property.

Diabetes is recognized as a world epidemic involving dysfunction of brain, eyes, heart, intestines, urogenital system and feet and therefore imposes an impact on the economy of many countries around the world. The principle of early recognition and complication prevention is widely recognized as optimal treatment. Treatment consists of administration of PDE5 inhibitors in a homogenized topical form mixed in slow-release type base to render a topical form.

Perivascular diabetes can lead to numerous associated disease states including but not limited to: diabetic foot ulcers and peripheral neuropathy; pulmonary hypertension; cardiovascular hypertension; aneurism; prostate disorders including cancer, benign prostatic hyperplasia, androgenic erectile dysfunction, transurethral resection of the prostate. Diabetic neuropathy has synonymously been referred to as peripheral arterial disease or Charcot neuropathy that subsequently leads to legs requiring amputations.

Patient Selection: 26 subjects, mostly diabetics (type 2) ranging in age group from 35 to 89 years on oral hypoglycemic agents. The duration of diabetes disease states that patients had been suffering from was between 10 days to 20 years. Patients who were chronic smokers, defined as smoking one pack/day for least 10 years, were suffering from peripheral arterial disease and one with accidental freezing from sub-zero temperature had peripheral arterial disease of 7 years in existence. The treatment period needed to restore a full resolution of circulation was from 3 days to 3 months.

The administration of Composition A involved applying on the areas with neuropathy, e.g. pain, pale or green coloration, cold intolerance and sensory loss, twice a day.

Patient selection was based on majority being primarily diabetics, in addition, chronic smokers were affected to some extent and select members with accidental impact e.g. freezing in sub-zero temperature or known vascular compromise in lower limb. The formulation of Composition A, through our literature search, has shown to be an innovative approach with the absence of any side effects. A diagnosis of peripheral arterial disease was established by symptoms recurrent pain in the feet with numbness, cold intolerance and change in the skin color to green or dark compared to remaining body.

Composition administration: 10-15 gm samples were given to the participants after obtaining their consent. A follow-up was conducted on a weekly basis or as needed. The duration of treatment required was decided by a full resolution of symptoms. The exact specifications and method of use of the topical treatment for prostatic hyperplasia and cancer prevention systems may vary upon manufacturing.

Results— Described in Table 1.

With the exceptions of 2 patients suffering from peripheral arterial disease for reasons other than diabetes it required on average up to 3 months of treatment to resolve the underlying perivascular disease—the treatment period ranged from 3 days to 4 months. A 57 year old female with an accidental freezing had a full resolution of symptoms in 3 months and those with nicotine addiction had a recovery from one to 3 months subject to smoking cessation. The success rate in majority was almost 99% with 78 yrs. Old with undiagnosed cause of peripheral arterial disease, likely multiple factors including smoking and a 95 years old male with chronic kidney disease and gout). A full resolution of symptoms was seen as normal coloration of skin on the feet, their normothermic response and absence of numbness. Some patients with initial ankle edema also had felt normal appearance in less than 3 months.

It is contemplated that therapeutic uses of the topical compositions may be used to treat prostatic hyperplasia, obstructive voiding, erectile dysfunction, and anorgasmic females. Other therapeutic uses, including androgenic response, Raynaud's Syndrome as a vasodilator, and cardio-vascular benefits are under review. It is envisaged that the composition be applied topically to affected areas twice a day.

Benign prostatic hypertrophy is of common occurrence in a medical practice, seen in men of advanced age after 50 years. It also leads to obstructive voiding and cancer. Determination of PSA levels have been the detection tool in the diagnosis. However, current studies have shown a lack of specificity. The relevance of hypertrophy or cancer in nephrology has been primarily, due to the presence of disease has to be ruled out in male kidney transplant donors.

Clinical research since 2016 on benign prostatic hyperplasia and cancer has been on searching for safer treatment options e.g. topical formulations consisting 2 of PDE5 inhibitors (such as papaverine and sildenafil). The trials at pilot studies have shown relief from obstructive voiding symptoms, decrease in PSA levels, increased cavernous blood flow, and androgenic effects without a PSA rise.

Current evidence has shown a decrease in pre-cancerous PC3 cells with papaverine. Current research is targeting the assessment of this parameter at monthly intervals of the PDE5 inhibitors. A current literature review from 2015 to date has shown significant evidence supporting the benefit from papaverine. Some authors have utilized injectable forms, however topical agents have been free of such complication.

The present invention advantageously fills the aforementioned deficiencies by providing a topical treatment for prostatic hyperplasia and cancer prevention. The present invention is superior to other systems in that it effectively eliminates the need for invasive surgery or the dangers associated with the use of multiple pharmaceuticals when treating prostatic hyperplasia.

The topical treatment for prostatic hyperplasia and cancer prevention systems may include a solution created with the base ingredients of homogenized papaverine hydrochloride USP and sildenafil citrate USP. Once the ingredients are combined, propylene glycol is poured atop them then warmed in intervals of ten seconds, with five to ten second pauses between warmings. Once the mixture is rendered to a clear solution, a PLO gel base is added to the solution then mixed until further homogeny occurs. The product may then be refrigerated for storing and eventual topical application to patients.

In one embodiment of the present invention, topical treatment for prostatic hyperplasia and cancer prevention systems may comprise a solution created with the base ingredients of homogenized papaverine hydrochloride USP and sildenafil citrate USP. Once the ingredients are combined, propylene glycol is poured a top them then warmed in intervals of ten seconds, with five to ten second pauses between warmings. Further, the mixture is then rendered to a clear solution and a PLO gel base is added to the solution then mixed until homogeny occurs.

### CONCLUSIONS

The topical compositions may be formulated to different consistencies by the use of different thickening agent. Semi solid formulations, such as creams, ointments, gels and pastes will provide different levels of occlusivity than more liquid preparations such as lotions and linaments. It is generally accepted that the higher the level of occlusion, the greater the level of transdermal delivery. Consequently, applying an occlusive dressing over the topical composition may increase the efficacy of the treatment.

The use of controlled release technologies will also increase local delivery to skin. The formulation itself may contain agents that control or increase the rate of release of the active agents, or a transdermal delivery system may be constructed to control the rate of diffusion of agents through the surface of the skin and into the systemic circulation. Transdermal patches similarly allow for systemic delivery of active agents. Transdermal delivery has many advantages: it is minimally painful, offers controlled release, has a lower rate of infection and higher rate of compliance compared to injections, and avoids first-pass metabolism seen in oral drug delivery. When transdermal delivery is used to provide systemic therapy, administration of the right dose depends on understanding the rate of drug penetration through the epidermis to the capillaries in the dermis and the properties of the active agents.

Embodiments of the present invention relate to topical treatment for various circulatory disorders. The various compositions may be used to treat disease states associated with perivascular diabetes including: diabetic foot ulcers; peripheral neuropathy; pulmonary hypertension; cardiovascular hypertension; Raynaud's Syndrome and aneurism. The various compositions may also be used to treat disease states associated with prostate disorders including: cancer; benign prostatic hyperplasia; androgenic erectile dysfunction; prostatic hyperplasia; cancer prevention; erectile dysfunction; obstructive bladder voiding; transurethral resection of the prostate; and anorgasmic females.

The foregoing descriptions of specific embodiments of the present invention have been presented for purposes of illustration and description. They are not intended to be exhaustive or to limit the invention and method of use to the precise forms disclosed. Obviously many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient, but is intended to cover the application or implementation without departing from the spirit or scope of the claims of the present invention.

What is claimed is:

1. A topical formulation comprising:
(i) a first active agent selected from at least one of papaverine, papaverine codecarboxylate, papaverine adenylate, papaverine teprosylate, and papaverine hydrochloride;
(ii) a second active agent selected from at least one of sildenafil, sildenafil HCl, sildenafil citrate, vardenafil, avanafil, tadalafil, and alprostadil;
(iii) a first compound or composition; and
(iv) a second compound or composition,
wherein the first compound or composition and second compound or composition are different, and each is selected from the group consisting of propylene glycol, polyvinyl alcohol; and Pluronic Lecithin Organogel, Lipoderm®, Lipmax™, Hydrogel, Medihol™, Oleabase™ Cryogel™, and Ointment Base.

2. The composition of claim 1, wherein the first active agent is papaverine hydrochloride.

3. The composition of claim 2, wherein the papaverine hydrochloride is present at a level of 10-20% (w/w).

4. The composition of claim 1, wherein the second active agent is sildenafil citrate.

5. The composition of claim 1, wherein the sildenafil citrate is present at a level of 10-20% (w/w).

6. The composition of claim 1, comprising:
papaverine hydrochloride U.S.P.;
sildenafil citrate U.S.P.;
propylene glycol U.S.P.; and
Pluronic Lecithin Organogel.

7. The composition of claim 6, wherein the papaverine hydrochloride is present at a level of 10-20% (w/w) and the sildenafil citrate is present at a level of 10-20% (w/w).

8. A method for treating disease states associated with perivascular diabetes including: diabetic foot ulcers; peripheral neuropathy; pulmonary hypertension; cardiovascular hypertension; aneurism; Raynaud's Syndrome and combinations thereof, comprising topically applying to skin, a composition comprising:
(i) a first active agent selected from at least one of papaverine, papaverine codecarboxylate, papaverine adenylate, papaverine teprosylate, and papaverine hydrochloride;
(ii) a second active agent selected from at least one of sildenafil, sildenafil HCl, sildenafil citrate, vardenafil, avanafil, tadalafil, and alprostadil;
(iii) a first compound or composition; and
(iv) a second compound or composition,
wherein the first compound or composition and second compound or composition are different, and each is selected from the group consisting of propylene glycol, polyvinyl alcohol; and Pluronic Lecithin Organogel, Lipoderm®, Lipmax™, Hydrogel, Medihol™, Oleabase™ Cryogel™, and Ointment Base.

9. The method of claim 8, wherein the composition comprises:
papaverine hydrochloride U.S.P.;
sildenafil citrate U.S.P.;
propylene glycol U.S.P.; and
Pluronic Lecithin Organogel.

10. The method of claim 8, wherein the papaverine hydrochloride U.S.P is present at a level of 10-20% (w/w).

11. The method of claim 8, wherein the sildenafil citrate U.S.P. is present at a level of 10-20% (w/w).

12. The method of claim 8, wherein the skin to be treated is subjected to electroporation prior to applying the composition to the skin.

13. The method of claim 8, wherein an occlusive dressing or transdermal delivery device is applied the over the topically applied formulation.

14. A method for treating prostate disorders including cancer, benign prostatic hyperplasia, androgenic erectile dysfunction, prostatic hyperplasia, cancer prevention, erectile dysfunction, obstructive bladder voiding, transurethral resection of the prostate, and combinations thereof, and anorgasmic females, comprising topically applying to skin, a composition comprising:
(i) a first active agent selected from at least one of papaverine, papaverine codecarboxylate, papaverine adenylate, papaverine teprosylate, and papaverine hydrochloride;
(ii) a second active agent selected from at least one of sildenafil, sildenafil HCl, sildenafil citrate, vardenafil, avanafil, tadalafil, and alprostadil;
(iii) a first compound or composition; and
(iv) a second compound or composition,
wherein the first compound or composition and second compound or composition are different, and each is selected from the group consisting of propylene glycol, polyvinyl alcohol; and Pluronic Lecithin Organogel, Lipoderm®, Lipmax™, Hydrogel, Medihol™, Oleabase™ Cryogel™, and Ointment Base.

15. The method of claim 14, wherein the composition comprises:
papaverine hydrochloride U.S.P.;
sildenafil citrate U.S.P.;
propylene glycol U.S.P.; and
Pluronic Lecithin Organogel.

16. The method of claim 15, wherein the papaverine hydrochloride U.S.P is present at a level of 10-20% (w/w).

17. The method of claim 15, wherein the sildenafil citrate U.S.P. is present at a level of 10-20% (w/w).

18. The method of claim 14, wherein the skin to be treated is subjected to electroporation prior to applying the composition to the skin.

19. The method of claim 14, wherein the skin to be treated is subjected to electroporation prior to applying the composition to the skin.

20. The method of claim 14, wherein an occlusive dressing or transdermal delivery device is applied the over the topically applied formulation.
